(19) United States
(12) Patent Application Publication
(10) Pub. No.: US 2014/0107121 A1
(43) Pub. Date: Apr. 17, 2014

(54) OLOPATADINE COMPOSITIONS AND USES THEREOF

(71) Applicant: Alcon Research, Ltd., Fort Worth, TX (US)

(72) Inventors: L. Wayne Schneider, Crowley, TX (US); Wesley Wehsin Han, Arlington, TX (US); Masood A. Chowhan, Arlington, TX (US)

(73) Assignee: Alcon Research, Ltd., Fort Worth, TX (US)

(21) Appl. No.: 14/108,432

(22) Filed: Dec. 17, 2013

Related U.S. Application Data
(63) Continuation of application No. 12/896,056, filed on Oct. 1, 2010.
(60) Provisional application No. 61/247,618, filed on Oct. 1, 2009.

(51) Int. Cl. A61K 31/5377; A61K 31/335; A61K 31/496

(52) U.S. Cl. CPC A61K 31/5377; A61K 31/496; A61K 31/335
USPC 514/235.2; 514/253.07

(57) ABSTRACT

The invention provides solution compositions comprising olopatadine and a PDE4 inhibitor compound of Formula I:

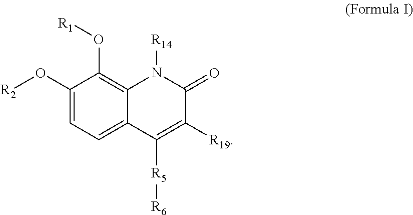

The invention also provides methods for treating allergic and inflammatory diseases. More particularly, the present invention relates to formulations of olopatadine and their use for treating and/or preventing allergic or inflammatory disorders of the eye, nose, skin, and ear.

### CROSS-REFERENCE TO RELATED APPLICATION

This application is a continuation of U.S. application Ser. No. 12/896,056 filed Oct. 1, 2010, and claims priority under 35 U.S.C. §119 to U.S. Provisional Patent Application No. 61/247,618 filed Oct. 1, 2009, the entire contents of which are incorporated herein by reference.

### FIELD OF THE INVENTION

The present invention relates to olopatadine formulations used for treating allergic and inflammatory diseases. More particularly, the present invention relates to formulations of olopatadine and their use for treating and/or preventing allergic or inflammatory disorders of the eye, ear, skin, and nose.

### BACKGROUND OF THE INVENTION

As taught in U.S. Pat. Nos. 4,871,865 and 4,923,892, both assigned to Burroughs Wellcome Co. (“the Burroughs Wellcome Patents”), certain carboxylic acid derivatives of doxepin, including olopatadine (chemical name: Z-11-(3-dimethylaminopropylidene)-6,11-dihydrodibenz[b,e]oxepine-2-acetic acid), have antihistamine and antiasthmatic activity. These two patents classify the carboxylic acid derivatives of doxepin as mast cell stabilizers with antihistaminic action because they are believed to inhibit the release of autacoids (i.e., histamine, serotonin, and the like) from mast cells and to inhibit directly histamine's effects on target tissues. The Burroughs Wellcome Patents teach various pharmaceutical formulations containing the carboxylic acid derivatives of doxepin, including nasal spray and ophthalmic formulations. See, for example, Col. 7, lines 7-26, and Examples 8 (H) and 8 (I) of the '865 patent.

U.S. Pat. No. 5,116,863, assigned to Kyowa Hakko Kogyo Co., Ltd., (“the Kyowa patent”), teaches that acetic acid derivatives of doxepin and, in particular, olopatadine, have anti-allergic and anti-inflammatory activity. Medicament forms taught by the Kyowa patent for the acetic acid derivatives of doxepin include a wide range of acceptable carriers; however, only oral and injection administration forms are mentioned.

U.S. Pat. No. 5,641,805, assigned to Alcon Laboratories, Inc. and Kyowa Hakko Kogyo Co., Ltd., teaches topical ophthalmic formulations containing olopatadine for treating allergic eye diseases. According to the '805 patent, the topical formulations may be solutions, suspensions or gels. The formulations contain olopatadine, an isotonic agent, and “if required, a preservative, a buffering agent, a stabilizer, a viscous vehicle and the like.” See Col. 6, lines 30-43. “[P]olyvinyl alcohol, polyvinylpyrrolidone, polyacrylic acid or the like” are mentioned as the viscous vehicle. See Col. 6, lines 55-57.

Phosphodiesterase type-IV (PDE4 or PDE-IV) is the predominant cyclic nucleotide hydrolyzing enzyme found in inflammatory leukocytes, such as mast cells, neutrophils, monocytes and T-lymphocytes. PDE4 inhibitor compounds are known to be useful as anti-inflammatory and anti-allergy agents.

In general, it is more desirable for active ingredients to be in solution rather than suspension in a pharmaceutical composition. For instance, solutions are easier to manufacture, easier to handle, provide better penetration to a target site of action, and provide better dosage consistency.

A formulation comprising both olopatadine and PDE4 inhibitor compounds is desirable because the combination addresses both the early and late phases of the allergic response. In addition, a formulation comprising compounds that enhance the solubility of olopatadine is desirable, because it assures that the olopatadine will not precipitate during a desired shelf life, and allows for an increased concentration of solubilized olopatadine.

### SUMMARY OF THE INVENTION

The invention provides pharmaceutical aqueous solution compositions comprising olopatadine and a PDE4 inhibitor compound of Formula I, as provided herein. The invention also provides methods for treating allergic and inflammatory conditions of the eye, ear, skin, and nose. In one aspect, the concentration of olopatadine is at least 0.17% w/v, and the concentration of the PDE4 inhibitor compound of Formula I is at least 0.05% w/v in a solution composition.

Specific preferred embodiments of the invention will become evident from the following more detailed description of certain preferred embodiments and the claims.

### BRIEF DESCRIPTION OF THE DRAWINGS

FIG. 1 is a graph showing Olopatadine Free Base Solubility versus PDE4 Inhibitor Concentration (% w/v).

FIG. 2 is a graph showing Olopatadine Free Base Solubility versus PDE4 Inhibitor Concentration (milliMolar).

### DETAILED DESCRIPTION OF THE INVENTION

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless indicated otherwise, all component amounts provided herein are presented on a % (w/v) basis and all references to olopatadine are to olopatadine free base.

In certain embodiments, the invention provides solution compositions comprising a therapeutically effective amount of olopatadine and a PDE4 inhibitor compound of Formula I that enhances the aqueous solubility of approximately 0.2-0.6% olopatadine.

The term “therapeutically effective amount” refers to the amount of a solution composition of the invention, olopatadine, or a PDE4 inhibitor compound of Formula I determined to produce a therapeutic response in a mammal Such therapeutically effective amounts are readily ascertained by one of ordinary skill in the art and using methods as described herein.

The terms “pharmaceutical aqueous solution composition” and “solution composition” as used herein refer to a composition comprising olopatadine or a pharmaceutically acceptable salt thereof, a PDE4 inhibitor compound of Formula I or a pharmaceutically acceptable salt thereof, and a pharmaceutically acceptable carrier (such as an ophthalmologic or nasal or otic carrier, or carrier suitable for delivery to the skin), excipient, or diluent as described herein that is capable of inducing a desired therapeutic effect (e.g. reducing, preventing, and/or eliminating allergies or allergy symptoms or inflammation) when properly administered to a patient. As used herein, the terms “pharmaceutical aqueous solution composition” and “solution composition” include compositions in which olopatadine (or a pharmaceutically acceptable salt thereof) and a PDE4 inhibitor compound of Formula I (or a pharmaceutically acceptable salt) are in solution, and wherein the overall composition is a solution, suspension, or semi-solid (for example cream, gel, or emulsion), depending on the presence or absence of any excipients in the composition.

As used herein, the term “pharmaceutically acceptable ophthalmologic or nasal or otic carrier” refers to those carriers that cause at most, little to no ocular, otic, or nasal irritation, provide suitable preservation if needed, and deliver olopatadine and a compound of Formula I in a homogenous dosage.

As used herein, the term “patient” includes human and animal subjects.

In one embodiment, a solution composition of the invention comprises a PDE4 inhibitor compound having structural Formula I:

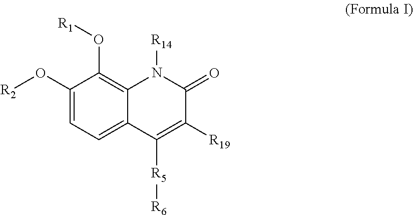

In certain embodiments:
R¹ and R² are independently selected from the group consisting of —(CH₂)ₛG¹G²G³, acyl, acylalkyl, carboxyalkyl, cyanoalkyl, alkoxy, alkoxyalkyl, amidoalkyl, amino, alkyl, alkylalkoxy, aminoalkyl, alkenyl, alkynyl, carboxyl, carboxyalkyl, ether, heteroalkyl, haloalkyl, cycloalkyl, cycloalkylalkyl, heterocycloalkyl, heterocycloalkylalkyl, aralkyl, aryl, guanidine, heteroaryl, heteroaralkyl, and hydroxyalkyl, any of which may be optionally substituted;
s is 1-8;
G¹ is selected from the group consisting of alkoxy, amino, amido, carbonyl, hydroxy, ether, an amino acid, and null;
G² is selected from the group consisting of alkyl, alkoxy, amino, aryl, halo, haloalkyl, heterocycloalkyl, heteroaryl, carboxylalkylamino, guanidine, an amino acid, and null, any of which may be optionally substituted;
G³ is selected from the group consisting of alkyl, alkoxy, amino, hydroxy, ether, carboxyl, hydroxamic acid, an amino acid, phosphonate, phosphoamide, and null, any of which may be optionally substituted;
R⁵ is selected from the group consisting of —(CR⁸R⁹)ₘW(CR¹⁰R¹¹)and —(CR¹²R¹³)ₚ—;
W is selected from the group consisting of O, N(R⁷), C(O)N(R⁷), and SO_{q};
m, n, and q are independently 0, 1 or 2;
p is 1 or 2;
R⁶ is selected from the group consisting of carboxyl, alkylcarboxy, amido, aryl, heteroaryl, cycloalkyl, heterocycloalkyl, alkyl, heteroalkyl, acyl, and hydroxamic acid, any of which may be optionally substituted;
R⁷ and R¹⁴ are independently selected from the group consisting of hydrogen, halogen, hydroxyl, lower alkyl, hydroxyalkyl, haloalkyl, and aminoalkyl;
R⁸, R⁹, R¹⁰, R¹¹, R¹² and R¹³ are independently selected from the group consisting of hydrogen and optionally substituted lower alkyl;
and R¹⁹ is selected from the group consisting of hydrogen, halogen, lower alkyl and haloalkyl; and
a pharmaceutically acceptable carrier or excipient.

In one embodiment, the PDE4 inhibitor compound of Formula I is (4-(3,5-Dichloropyridin-4-ylamino)-7-methoxy-8-(6-(4-methylpiperazin-1-yl)hexyloxy)quinolin-2(1H)-one):

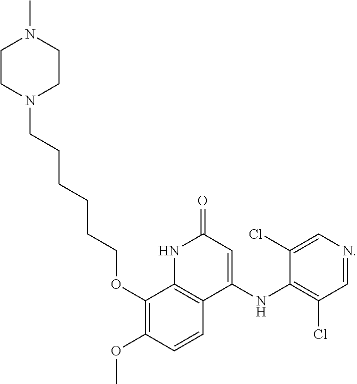 [SMILES: COc1ccc2c(Nc3c(Cl)cncc3Cl)cc(=O)[nH]c2c1OCCCCCCN1CCN(C)CC1]

In another embodiment, the compound of Formula I is (4-(3,5-Dichloropyridin-4-ylamino)-7-methoxy-8-(6-morpholinohexyloxy)quinolin-2(1H)-one):

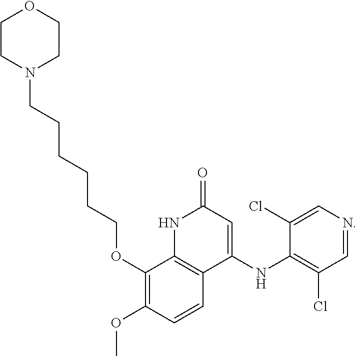 [SMILES: COc1ccc2c(Nc3c(Cl)cncc3Cl)cc(=O)[nH]c2c1OCCCCCCN1CCOCC1]

These compounds, and other PDE4 inhibitor compounds of Formula I, are PDEIV inhibitors, and are described in detail in co-pending U.S. application Ser. No. 11/774,053 filed Jul. 6, 2007, and U.S. application Ser. No. 12/544,185 filed Aug. 19, 2009, the disclosures of which are incorporated by reference in their entirety.

Olopatadine is a known compound that can be obtained by the methods disclosed in U.S. Pat. No. 5,116,863, the entire contents of which are hereby incorporated by reference in the present specification.

Generally, olopatadine will be added in the form of a pharmaceutically acceptable salt. Examples of the pharmaceutically acceptable salts of olopatadine include inorganic acid salts such as hydrochloride, hydrobromide, sulfate and phosphate; organic acid salts such as acetate, maleate, fumarate, tartrate and citrate; alkali metal salts such as sodium salt and potassium salt; alkaline earth metal salts such as magnesium salt and calcium salt; metal salts such as aluminum salt and zinc salt; and organic amine addition salts such as triethylamine addition salt (also known as tromethamine), morpholine addition salt and piperidine addition salt. The most preferred form of olopatadine for use in the solution compositions of the present invention is the hydrochloride salt of (Z)-11-(3-dimethylaminopropylidene)-6,11-dihydrodibenz-[b,e]oxepin-2-acetic acid. When olopatadine is added to the compositions of the present invention in this salt form, 0.222% olopatadine hydrochloride is equivalent to 0.2% olopatadine free base, 0.443% olopatadine hydrochloride is equivalent to 0.4% olopatadine free base, and 0.665% olopatadine hydrochloride is equivalent to 0.6% olopatadine free base. As used herein, reference to a concentration of olopatadine refers to olopatadine free base concentration, unless otherwise specified.

The PDE4 inhibitor compounds of Formula I have been unexpectedly found to increase the solubility of olopatadine. Thus, an aqueous solution composition of the present invention can be prepared without the need for any other solubility enhancing components.

The compositions administered according to the present invention may also include various other ingredients, including but not limited to surfactants, tonicity agents, buffers, preservatives, and viscosity building agents.

An appropriate buffer system (e.g., sodium phosphate, sodium acetate, sodium citrate, sodium borate or boric acid) may be added to the compositions to prevent pH drift under storage conditions. The particular concentration will vary, depending on the agent employed. Preferably, however, the buffer will be chosen to maintain a target pH within the range of pH 6.0-7.5.

In certain embodiments, the concentration of olopatadine in a solution composition of the invention is at least 0.05% w/v. For example, the concentration of olopatadine can be about 0.05%, 0.075%, 0.10%, 0.15%, 0.20%, 0.25%, 0.30%, 0.35%, 0.40%, 0.45%, 0.50%, 0.55%, or 0.60% w/v, or higher. In certain embodiments, a solution composition of the invention is a solution formulation that contains at least 0.05% w/v olopatadine. In certain embodiments, solution formulations of the present invention contain 0.17-0.62% w/v olopatadine. In certain embodiments, solution formulations intended for use in the eye contain 0.17-0.25% olopatadine, and preferably 0.18-0.22% w/v olopatadine. In certain embodiments, solution formulations intended for use in the nose contain 0.38-0.62% w/v olopatadine.

In certain embodiments, the concentration of a PDE4 inhibitor compound of Formula I in a solution composition of the invention is at least 0.05% w/v. For example, the concentration of a PDE4 inhibitor compound of Formula I can be about 0.05%, 0.10%, 0.15%, 0.20%, 0.25%, 0.30%, 0.35%, 0.40%, 0.45%, 0.50%, 0.55%, or 0.60% w/v, or higher.

In certain embodiments, solution compositions of the invention are useful for treating allergic or inflammatory disorders, including allergic or inflammatory disorders of the eye, nose, skin, and ear.

In certain embodiments, an ophthalmic formulation is administered to the eye of a patient in need thereof to treat an ocular disorder. The term “ocular disorder” as used herein includes allergic and/or inflammatory conditions of the eye, such as ophthalmic allergic disorders, including allergic conjunctivitis, vernal conjunctivitis, vernal keratoconjunctivitis, and giant papillary conjunctivitis, dry eye, glaucoma, corneal neovascularization, optic neuritis, Sjogren's syndrome, retinal ganglion degeneration, ocular ischemia, retinitis, retinopathies, uveitis, ocular photophobia, and of inflammation and pain associated with acute injury to the eye tissue. Specifically, the compounds may be used to treat glaucomatous retinopathy and/or diabetic retinopathy. The compounds may also be used to treat post-operative inflammation or pain as from ophthalmic surgery such as cataract surgery and refractive surgery. In certain embodiments, the compounds of the present invention are used to treat an allergic eye disease selected from the group consisting of allergic conjunctivitis; vernal conjunctivitis; vernal keratoconjunctivitis; and giant papillary conjunctivitis. allergic conjunctivitis.

In one embodiment, a solution composition of the invention is an ophthalmic formulation for delivery to the eye, such as a topical ophthalmic formulation. The solution composition may comprise ophthalmologically acceptable preservatives, surfactants, viscosity enhancers, penetration enhancers, buffers, tonicity agents, and water to form an aqueous, sterile ophthalmic solution, suspension, or emulsion. Gelling agents can also be used, including, but not limited to, gellan and xanthan gum. In order to prepare sterile ophthalmic ointment formulations, olopatadine and a PDE4 inhibitor compound of Formula I are combined with a preservative in an appropriate vehicle. Sterile ophthalmic gel formulations may be prepared by suspending olopatadine and a PDE4 inhibitor compound of Formula I in a hydrophilic base prepared from the combination of, for example, CARBOPOL®-974, CARBOPOL®-940 (BF Goodrich, Charlotte, N.C.), or the like, according to the published formulations for analogous ophthalmic preparations; preservatives and tonicity agents can be incorporated.

Solution compositions of the invention can be administered topically to the eye, for example, to treat allergic conjunctivitis and/or ocular inflammation. In general, the doses used for the above described purposes will vary, but will be in an effective amount to reduce or eliminate allergic conjunctivitis and/or ocular inflammation. Generally, 1-2 drops of such compositions will be administered one or more times per day. For example, the composition can be administered 2 to 3 times a day or as directed by an eye care provider.

Topical ophthalmic products may also be packaged in multidose form. Preservatives may thus be required to prevent microbial contamination during use. Suitable preservatives include: benzalkonium chloride, benzododecinium bromide, chlorobutanol, methyl paraben, propyl paraben, phenylethyl alcohol, edetate disodium, sorbic acid, polyquaternium-1, or other agents known to those skilled in the art. Such preservatives are typically employed at a level of from 0.001 to 5.0% w/v. Unit dose compositions of the present invention will be sterile, but typically unpreserved. Such compositions, therefore, generally will not contain preservatives. The ophthalmic compositions of the present invention may also be provided preservative free and packaged in unit dose form.

The compositions of the present invention optionally comprise one or more excipients. Excipients commonly used in solution compositions intended for topical application to the eyes or nose, such as solutions or sprays, include, but are not limited to, tonicity agents, preservatives, chelating agents, buffering agents, surfactants and antioxidants. Suitable tonicity-adjusting agents include mannitol, sodium chloride, glycerin, sorbitol and the like. Suitable preservatives include p-hydroxybenzoic acid ester, benzalkonium chloride, benzododecinium bromide, polyquaternium-1 and the like. Suitable chelating agents include sodium edetate and the like. Suitable buffering agents include phosphates, borates, citrates, acetates, tromethamine, and the like. Suitable surfactants include ionic and nonionic surfactants, though nonionic surfactants are preferred, such as polysorbates, polyethoxylated castor oil derivatives, polyethoxylated fatty acids, polyethoxylated alcohols, polyoxyethylene-polyoxypropylene block copolymers, and oxyethylated tertiary octylphenol formaldehyde polymer (tyloxapol). Suitable antioxidants include sulfites, thiosulfate, ascorbates, BHA, BHT, tocopherols, and the like. The compositions of the present invention optionally comprise an additional active agent. The compositions of the present invention may contain one or more nonionic, anionic, or cationic polymers as lubricants or as viscosity agents, including but not limited to hydroxypropyl methylcelluloses (HPMCs), methylcelluloses, carboxymethylcelluloses (CMCs), polyethylene glycols (PEGs), poloxamers, polypropylene glycols, xanthan gums, guar gums, carbomers, polyvinyl alcohols (PVAs), polyvinylpyrrolidones (PVPs), alginic acids and salts, gellan gums, carrageenans, and chitosans.

Various tonicity agents may be employed to adjust the tonicity of the composition, preferably to that of natural tears for ophthalmic compositions. For example, sodium chloride, potassium chloride, magnesium chloride, calcium chloride, dextrose, mannitol, sorbitol, propylene glycol, or glycerol may be added to the composition to approximate physiological tonicity. Such an amount of tonicity agent will vary, depending on the particular agent to be added. In general, however, the compositions will have a tonicity agent in an amount sufficient to cause the final composition to have an ophthalmically acceptable osmolality (generally about 150-450 mOsm, preferably 250-350 mOsm).

In certain embodiments, a composition of the invention has a pH of about 3.0 to about 8.5. In one embodiment, an ophthalmic composition of the present invention has a pH of 4.0-8.0, preferably a pH of 5.0-7.5, and most preferably a pH of 6.0-7.4. Compositions of the present invention intended for use in the nose preferably have a pH of 3.0-8.0 and most preferably a pH of 5.0-7.5.

In certain embodiments, a solution composition of the invention can be formulated for nasal applications, and can be used to treat nasal disorders. Thus, in certain embodiments, the invention provides methods for treating a nasal disorder, comprising administering a solution composition of the invention to the nose of a patient in need thereof The term “nasal disorder” as used herein includes allergic and/or inflammatory conditions of the nose.

In a further embodiment, nasal solution compositions of the invention are formulated to provide for a therapeutically effective intranasal concentration. For example, a nasal solution composition of the invention may have an intranasal concentration of about 0.1-1000 nM or 1-100 nM. Intranasal compositions are delivered to the nasal mucosa one to four times per day according to the routine discretion of a skilled clinician. The pH of the formulation should range from 3 to 8 or preferably from 5 to 7.5. Topical administration directly onto the nasal mucosa via an intranasal insert or implant device or a solution drug-delivery-sponge (GELFOAM®, Pharmacia & Upjohn, Kalamazoo, Mich.) may deliver olopatadine and a PDE4 inhibitor compound of Formula I at the rate of 1-2 μl/hour (e.g. 0.0001-10 mg/day) for several weeks according to the device design, its drug release characteristics, and according to the discretion of a skilled clinician.

While the precise regimen is left to the discretion of the clinician, the resulting solution or solutions are preferably administered intranasally as described herein one to four times a day, or as directed by the clinician.

A nasally acceptable carrier refers to those carriers that cause at most, little to no nasal irritation, provide suitable preservation if needed, and deliver a solution composition of the present invention in a homogenous dosage. For nasal delivery, a solution composition of the invention may be combined with nasally acceptable preservatives, co-solvents, surfactants, viscosity enhancers, penetration enhancers, buffers, tonicity agents, and water to form an aqueous, sterile suspension, solution, emulsion, or viscous, semi-viscous, or semi-solid gels. Nasal solution formulations may be prepared by dissolving the agent in a physiologically acceptable isotonic aqueous buffer. Further, the nasal solution may include a nasally acceptable surfactant. Viscosity building compounds, such as hydroxymethyl cellulose, hydroxyethyl cellulose, methylcellulose, or carbomers, for example, may be added to the compositions of the present invention to improve the retention of the compounds.

In order to prepare a sterile nasal ointment formulation, a solution composition of the invention may comprise a preservative in an appropriate vehicle. Sterile nasal gel formulations may be prepared by suspending olopatadine and/or the PDE4 inhibitor compound of Formula I in a hydrophilic base prepared from, for example, CARBOPOL®-974, CARBOPOL®-940 (BF Goodrich, Charlotte, N.C.), or the like, according to methods known in the art for other suitable nasal formulations. VISCOAT® (Alcon Laboratories, Inc., Fort Worth, Tex.) may be used for intranasal injection, for example. Other compositions of the present invention may contain penetration enhancing materials such as CREMOPHOR® (Polyoxyethylene castor oil) and TWEEN® 80 (polyoxyethylene sorbitan monolaureate).

The compositions of the invention can be administered intranasally in the form of a nasal spray, as is known to those skilled in the art.

Nasal delivery may be achieved by incorporation of olopatadine and the PDE4 inhibitor compound of Formula I into bioadhesive particulate carriers (<200 μm) such as those comprising cellulose, polyacrylate or polycarbophil, in conjunction with suitable absorption enhancers such as phospholipids or acylcarnitines. Available systems include those developed by DanBiosyst and Scios. The formulation can be administered using a simple nasal spray device available from companies such as Valois or Pfeiffer.

In certain embodiments, a solution composition comprising olopatadine and a PDE4 inhibitor compound of Formula I is formulated for delivery to the skin. Particularly compositions intended for application to the skin can be solution, suspension or semisolid. However, the olopatadine (or pharmaceutically acceptable salt thereof) and PDE4 inhibitor compound (or pharmaceutically acceptable salt thereof) presented in the said dosage forms should be all molecularly dissolved as a solution. The excipients presented in the dosage forms can be solid as a suspension or semisolid as a cream, for example. The viscosity of the said compositions can be variant from 1 to 100,000 cps or higher depending on the needs of the dermatological product.

In a further embodiment, otic compositions comprising olopatadine and a PDE4 inhibitor compound of Formula I are formulated to provide for a pharmacologically effective intraotic concentration. Topical otic compositions may be delivered to the ear one to four or more times per day according to the routine discretion of a skilled clinician. The pH of the formulation should range from 4.0 to 9.0, or from 4.5 to 7.4. Topical administration directly onto the otic nerves (auditory and vestibular) and/or otic nerve-heads via an intraotic insert or implant device or a solution drug-delivery-sponge (GELFOAM®, Pharmacia & Upjohn, Kalamazoo, Mich.) may deliver a solution composition of the invention at the rate of 1-2 μl/hour (e.g. 0.0001-10 mg/day) for several weeks according to the device design, its drug release characteristics, and according to the discretion of a skilled clinician.

For otic delivery, a solution composition of the invention may be combined with otically acceptable preservatives, co-solvents, surfactants, viscosity enhancers, penetration enhancers, buffers, tonicity agents, or water to form an aqueous, sterile suspension, solution, or viscous, semi-viscous, or semi-solid gels.

Solution compositions of the present invention may be delivered directly to the ear (for example: topical otic drops or ointments; slow release devices in the ear or implanted adjacent to the ear). Local administration includes otic intramuscular, intratympanic cavity and intracochlear injection routes of administration. Furthermore, a solution composition of the invention can be administered to the inner ear by placement of a gelfoam, or similar absorbent and adherent product, soaked with a solution composition of the invention against the window membrane of the middle/inner ear or adjacent structure with due discretion and caution by a skilled clinician.

The compositions of the present invention are preferably packaged in opaque plastic containers. A preferred container for an ophthalmic product is a low-density polyethylene container that has been sterilized using ethylene oxide instead of gamma-irradiation. A preferred container for a nasal product is a high-density polyethylene container equipped with a nasal spray pump.

The references cited herein, to the extent that they provide exemplary procedural or other details supplementary to those set forth herein, are specifically incorporated by reference.

Unless otherwise required by context, singular terms used herein shall include pluralities and plural terms shall include the singular.

### EXAMPLES

The following examples, including the experiments conducted and results achieved are provided for illustrative purposes only and are not to be construed as limiting the invention.

### Example 1

### Olopatadine-PDE4 Inhibitor Solubility Study

The following experiments were conducted to determine the effect of compounds of Formula I on the aqueous solubility of olopatadine.

Formulations with the compositions shown in Table 1 were prepared for olopatadine solubility testing as follows: ten milliliter samples of the formulations containing either 0%, 0.1%, 0.3%, or 1% of either Compound 1 (4-(3,5-Dichloropyridin-4-ylamino)-7-methoxy-8-(6-(4-methylpiperazin-1-yl)hexyloxy)quinolin-2(1H)-one) or Compound 2 (4-(3,5-Dichloropyridin-4-ylamino)-7-methoxy-8-(6-morpholinohexyloxy)quinolin-2(1H)-one) with at least 1% Olopatadine Hydrochloride as shown in Table 2 were prepared and adjusted to the target pH. Compound 2 was not tested at pH 7.4 as it was not sufficiently soluble at that pH. The samples were mixed on a rocker and the pH was readjusted to the target pH after one and six days of mixing. On day seven, the samples were filtered through an Acrodisc 25 mm GXF/GHP 0.2 micron filter. The first three milliliters of filtrate were collected for final pH measurement and the next 3 milliliters of filtrate were filled into two 1.5 mL HPLC vials for the olopatadine assay (as free base) as shown below. Compound 1 was not assayed in samples A through H as an assay method was not available. Duplicate Compound 1/Olopatadine samples for the olopatadine assay were injected neat into the HPLC. Compound 2 and olopatadine were assayed in samples I, J, and K. Single Compound 2/Olopatadine samples were diluted 1/10 with 50/50 Acetonitrile/Water and injected into the UPLC.

TABLE 1
General Formulation used for Olopatadine-
PDE4 Inhibitor Solubility Studies

| Ingredient | Target Concentration |
|---|---|
| Olopatadine Hydrochloride | >1% (i.e., saturated) |
| Compound 1 or Compound 2 | 0%, 0.1%, 0.3% or 1% |
| Sodium Hydroxide and/or Hydrochloric Acid | q.s. pH 5.2 or 7.4 |
| Polyquaternium-1 | 0.001% |
| Boric Acid | 0.6% |
| Mannitol | 0.3% |
| Sodium Chloride | 0.5% |
| Purified Water | q.s. 100% |

The final pH of the filtered samples was measured with an Orion 525A+pH meter using a Ross Semimicro combination pH electrode and automatic temperature probe.

The Compound 1 and Olopatadine HPLC assay was conducted using the following conditions:
Instrument: Waters 2695 Separation Module and Waters 2487
Variable Wavelength Ultraviolet-Visible Detector with Empower Software
Column: Phenomenex Ultracarb C8, 5 micron, 150×4.6 mm
Mobile Phase:
   Solvent A=Acetonitrile
   Solvent B=100 milliMolar Potassium Phosphate with 0.1% Triethylamine adjusted to pH 3.0 with NaOH/HC1
   Flowrate=1 milliliter/Minute
   Gradient:

| Time (min) | % A | % B | |
|---|---|---|---|
| 0 | 28 | 72 | |
| 11 | 50 | 50 | |
| 22 | 50 | 50 | |
| 23 | 28 | 72 | |
| 30 | 28 | 72 | End of injection-run. |

Detection: 299 nm Ultraviolet Absorbance
Injection Volume: 20 microliters
Olopatadine Retention Time: About 6.2 minutes

The Compound 2 and Olopatadine UPLC assay was conducted using the following conditions:
Instrument: Waters ACQUITY UPLC System with TUV Detector and Empower Software
Column: Acquity UPLC BEH Shield C18, 1.7 micron, 100×2.1 mm
Mobile Phase:
   Solvent A=0.1% Phosphoric Acid adjusted to pH 3.0 with NaOH/HCl
   Solvent B=Acetonitrile
   Flowrate=0.3 milliliter/Minute
   Gradient:

| Time (min) | % A | % B | |
|---|---|---|---|
| 0 | 75 | 25 | |
| 8.5 | 20 | 80 | |
| 9 | 75 | 25 | |
| 14 | 75 | 25 | End of injection-run. |

Detection: 285 nm Ultraviolet Absorbance
Injection Volume: 3 microliters
AL-53817 Retention Time: About 4.1 minutes
Olopatadine Retention Time: About 4.5 minutes

The final filtrate pHs, olopatadine and Compound 2 HPLC assay results, and target Compound 1 concentrations for the samples are shown in Table 2.

TABLE 2
Results of Olopatadine-PDE4 Solubility Studies

| | | | | Olopatadine Solubility as Free Base | | | |
|---|---|---|---|---|---|---|---|
| Sam- | | Compound 1 | | % w/v | % w/v | | |
| ple | Final | Conc. | | Sam- | Samp- | Ave | |
| Code | pH | % w/v | mM | ple1 | le2 | % w/v | mM |
| A | 7.41 | 0 | 0.00 | 0.17874 | 0.17908 | 0.179 | 5.30 |
| B | 7.43 | 0.1 | 1.87 | 0.21301 | 0.21327 | 0.213 | 6.32 |
| C | 7.41 | 0.3 | 5.61 | 0.27198 | 0.27233 | 0.272 | 8.07 |
| D | 7.43 | 1 | 18.71 | 0.46740 | 0.46729 | 0.467 | 13.85 |
| E | 5.41 | 0 | 0.00 | 0.20068 | 0.20077 | 0.201 | 5.95 |
| F | 5.40 | 0.1 | 1.87 | 0.23711 | 0.23688 | 0.237 | 7.02 |
| G | 5.36 | 0.3 | 5.61 | 0.30268 | 0.30232 | 0.303 | 8.97 |
| H | 5.31 | 1 | 18.71 | 0.54933 | 0.54847 | 0.549 | 16.27 |
| | | Compound 2 | | | | | |
| Code | pH | Conc. | | Olopatadine Solubility as Free Base | | | |
| E | 5.41 | 0 | 0.00 | 0.20068 | 0.20077 | 0.201 | 5.95 |
| I | 5.42 | 0.0907 | 1.74 | 0.23037 | NA | 0.230 | 6.83 |
| J | 5.43 | 0.2818 | 5.40 | 0.29275 | NA | 0.293 | 8.68 |
| K | 5.34 | 0.973 | 18.66 | 0.52087 | NA | 0.521 | 15.44 |

The target pH of samples E through K was 5.2 and the pH readings of the suspensions were close to this value prior to filtration. However, after filtration the solution pH was generally about 0.2 pH units higher than the suspension pH. This pH shift is commonly observed when measuring the pH of a suspension versus a solution. Duplicate samples of A through H were assayed and the duplicate values were averaged.

The milliMolar (mM) concentrations were calculated by dividing the % w/v concentrations by the molecular weight and multiplying by 10000.

The molecular weights were as follows:

Olopatadine (as free base)=337.4 g/mole;

Compound 1=534.5 g/mole;

Compound 2=521.4 g/mole.

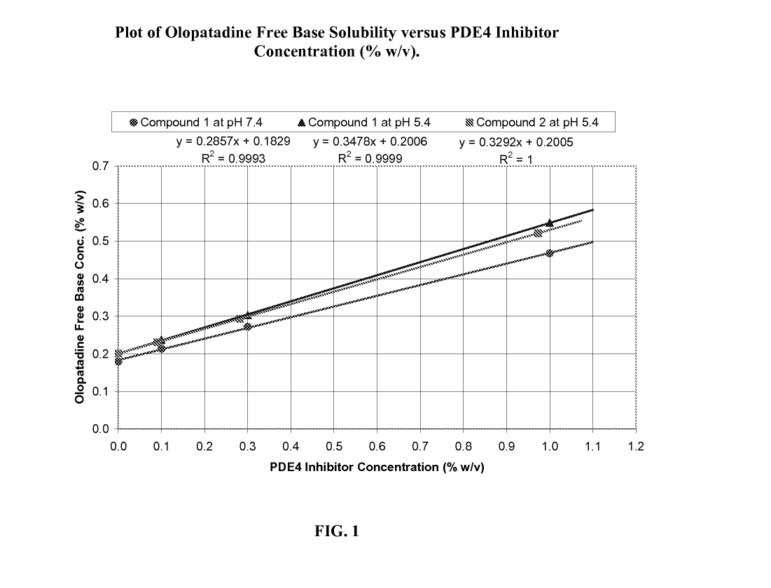

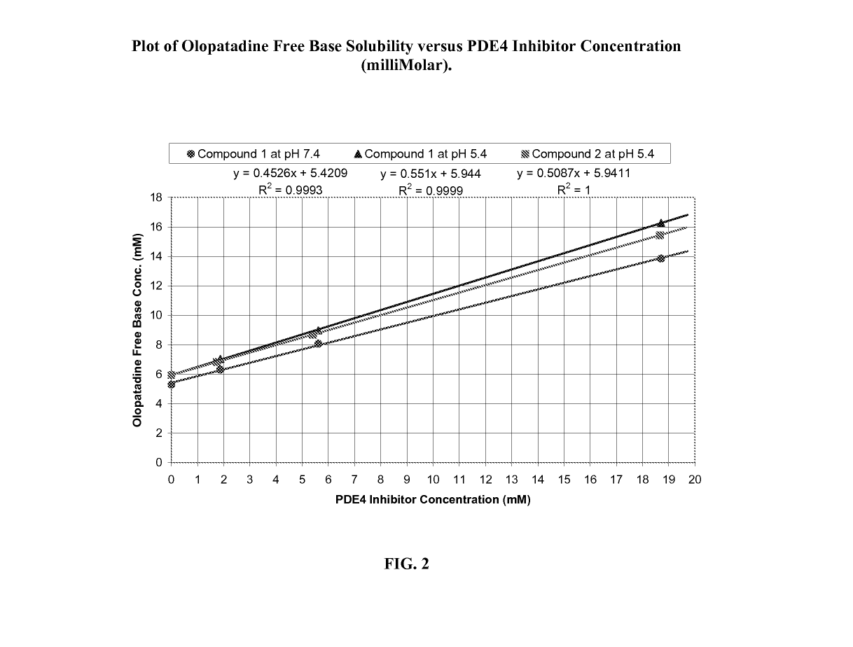

The concentrations of Compounds 1 and 2 were plotted against the resulting Olopatadine free base solubilities as % w/v and milliMolar (mM) concentrations and a straight line equation was fit to the data (FIGS. 1 and 2).

Both Compound 1 and Compound 2 increased the aqueous solubility of olopatadine in a linear concentration dependent manner. The ratio of solubility enhancement was about two molecules of the Compounds to one olopatadine molecule.

It should be understood that the foregoing disclosure emphasizes certain specific embodiments of the invention and that all modifications or alternatives equivalent thereto are within the spirit and scope of the invention as set forth in the appended claims.

What is claimed is:

1. A pharmaceutical aqueous solution composition comprising:
a therapeutically effective amount of olopatadine or a pharmaceutically acceptable salt thereof as a soluble form in the aqueous phase,
a PDE4 inhibitor compound of Formula I, 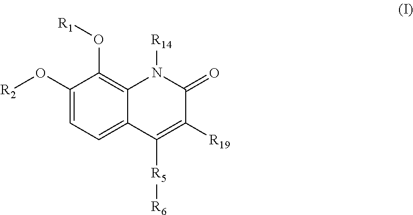
or a pharmaceutically acceptable salt thereof, wherein:
R¹ and R² are independently selected from the group consisting of —(CH₂)ₛG¹G²G³, acyl, acylalkyl, carboxyalkyl, cyanoalkyl, alkoxy, alkoxyalkyl, amidoalkyl, amino, alkyl, alkylalkoxy, aminoalkyl, alkenyl, alkynyl, carboxyl, carboxyalkyl, ether, heteroalkyl, haloalkyl, cycloalkyl, cycloalkylalkyl, heterocycloalkyl, heterocycloalkylalkyl, aralkyl, aryl, guanidine, heteroaryl, heteroaralkyl, and hydroxyalkyl, any of which may be optionally substituted;
s is 1-8;
G¹ is selected from the group consisting of alkoxy, amino, amido, carbonyl, hydroxy, ether, an amino acid, and null;
G² is selected from the group consisting of alkyl, alkoxy, amino, aryl, halo, haloalkyl, heterocycloalkyl, heteroaryl, carboxylalkylamino, guanidine, an amino acid, and null, any of which may be optionally substituted;
G³ is selected from the group consisting of alkyl, alkoxy, amino, hydroxy, ether, carboxyl, hydroxamic acid, an amino acid, phosphonate, phosphoamide, and null, any of which may be optionally substituted;
R⁵ is selected from the group consisting of —(CR⁸R⁹)ₘW(CR¹⁰R¹¹)ₙ— and —(CR¹²R¹³)ₚ—;
W is selected from the group consisting of O, N(R⁷), C(O)N(R⁷), and SO_{q};
m, n, and q are independently 0, 1 or 2;
p is 1 or 2;
R⁶ is selected from the group consisting of carboxyl, alkylcarboxy, amido, aryl, heteroaryl, cycloalkyl, heterocycloalkyl, alkyl, heteroalkyl, acyl, and hydroxamic acid, any of which may be optionally substituted;
R⁷ and R¹⁴ are independently selected from the group consisting of hydrogen, halogen, hydroxyl, lower alkyl, hydroxyalkyl, haloalkyl, and aminoalkyl;
R⁸, R⁹, R¹⁰, R¹¹, R¹² and R¹³ are independently selected from the group consisting of hydrogen and optionally substituted lower alkyl;
and R¹⁹ is selected from the group consisting of hydrogen, halogen, lower alkyl and haloalkyl; and
a pharmaceutically acceptable carrier or excipient,
wherein the concentration of olopatadine in the solution composition is at least 0.17% w/v.

2. The solution composition of claim 1, wherein the PDE4 inhibitor compound is (4-(3,5-Dichloropyridin-4-ylamino)-7-methoxy-8-(6-(4-methylpiperazin-1-yl)hexyloxy)quinolin-2(1H)-one or (4-(3,5-Dichloropyridin-4-ylamino)-7-methoxy-8-(6-morpholinohexyloxy)quinolin-2(1H)-one).

3. The solution composition of claim 2, wherein the PDE4 inhibitor compound is (4-(3,5-Dichloropyridin-4-ylamino)-7-methoxy-8-(6-(4-methylpiperazin-1-yl)hexyloxy)quinolin-2(1H)-one.

4. The solution composition of claim 2, wherein the PDE4 inhibitor compound is (4-(3,5-Dichloropyridin-4-ylamino)-7-methoxy-8-(6-morpholinohexyloxy)quinolin-2(1H)-one).

5. The solution composition of claim 1, wherein the concentration of olopatadine is 0.17-0.62% w/v.

6. The solution composition of claim 5, wherein the concentration of olopatadine is 0.17-0.25% w/v.

7. The solution composition of claim 6, wherein the concentration of olopatadine is 0.18-0.22% w/v

8. The solution composition of claim 1, wherein the concentration of the PDE4 inhibitor compound of Formula 1 is at least 0.05% w/v.

9. The solution composition of claim 1, wherein the concentration of the PDE4 inhibitor compound of Formula 1 is at least 0.1% w/v

10. The solution composition of claim 1 having a pH in the range of 3.0 to 8.0.

11. The solution composition of claim 10, wherein the pH is in the range of 5.0 to 7.5.

12. The solution composition of claim 11, wherein the pH is in the range of 6.0 to 7.4.

13. The solution composition of claim 1, wherein the composition is formulated for delivery to the eye, nose, or skin.

14. The solution composition of claim 13, wherein the solution composition is formulated for delivery to the skin and the solution composition is a viscous solution or a gel.

15. A method for treating an allergic or inflammatory condition of the eye, nose, or skin, comprising administering a pharmaceutically effective amount of the solution composition of claim 1 to a patient in need thereof.
